(19) United States
(12) Patent Application Publication
(10) Pub. No.: US 2008/0050428 A1
(43) Pub. Date: Feb. 28, 2008

(54) Oral Pharmaceutical Preparation Comprising a Proton Pump Antagonist and a Basic Excipient

(75) Inventors: Hartmut Ney, Konstanz (DE); Isabel Anstett-Klein, Leinfelden-Echterdingen (DE); Simone Hiltl, Konstanz (DE); Antje Brueck-Scheffler, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 11/663,998

(22) PCT Filed: Sep. 30, 2005
(86) PCT No.: PCT/EP05/54956

§ 371 Date: Apr. 30, 2007

(30) Foreign Application Priority Data
Oct. 5, 2004 (EP) 04104884.4

(51) Int. Cl. A61K 31/4375; A61K 9/20; A61P 1/04

(52) U.S. Cl. 424/464; 514/301

(57) ABSTRACT

The invention relates to novel extended release dosage forms for reversible proton pump antagonists comprising a basic excipient.

### TECHNICAL FIELD

The present invention relates to oral pharmaceutical preparations in multiparticulate form or in tablet form for proton pump antagonists.

### STATE OF THE ART

Irreversible proton pump inhibitors (H⁺/K⁺-ATPase inhibitors, PPIs), especially pyridin-2-ylmethyl-sulphinyl-1H-benzimidazoles as disclosed for example in EP-A-0 005 129, EP-A-0 166 287, EP-A-0 174 726 and EP-A-0 268 956, have, by reason of their H⁺/K⁺-ATPase-inhibiting effect, importance in the therapy of diseases derived from increased gastric acid secretion. Irreversible proton pump inhibitors are substances which bind covalently, and thus irreversibly, to the enzyme responsible for acid secretion in the stomach, the H⁺/K⁺-ATPase [description of the mechanism of action for example in Wurst et al., The Yale Journal of Biology and Medicine 69, (1996), 233-243]. Examples of commercially available active ingredients from this group are 5-methoxy-2-[(4-methoxy-3,5-dimethyl-2-pyridinyl)methylsulphinyl]-1H-benzimidazole (INN: omeprazole), 5-difluoromethoxy-2-[(3,4-dimethoxy-2-pyridinyl)methylsulphinyl]-1H-benzimidazole (INN: pantoprazole), 2-[3-methyl-4-(2,2,2-trifluoroethoxy)-2-pyridinyl)methylsulphinyl]-1H-benzimidazole (INN: lansoprazole) and 2-{[4-(3-methoxypropoxy)-3-methylpyridin-2-yl]methylsulphinyl}-1H-benzimidazole (INN: rabeprazole).

Besides the so-called irreversible proton pump inhibitors which, as mentioned at the outset, essentially have a common basic chemical structure (they are pyridinylmethylsulphinylbenzimidazoles), there are so-called reversible H⁺/K⁺-ATPase inhibitors which have different basic chemical structures and which—as indicated by the name—reversibly bind to the enzyme responsible for gastric acid secretion and are therefore also called proton pump antagonists or APAs (=acid pump antagonists) [description of the mechanism of action for example in Wurst et al, The Yale Journal of Biology and Medicine 69 (1996), 233-243]. Reversible proton pump inhibitors are disclosed for example in the documents DE-A 3917232, EP-A-0399267, EP-A-0387821, JP-A-3031280, JP-A-2270873, EP-A-0308917, EP-A-0268989, EP-A-0228006, EP-A-0204285, EP-A-0165545, EP-A-0125756, EP-A-0120589, EP-A-0509974, DE-A 3622036, EP-A-0537532, EP-A-0535529, JP-A-3284686, JP-A-3284622, U.S. Pat. No. 4,833,149, EP-A-0261912, WO-A-9114677, WO-A-9315055, WO-A-9315071, WO-A-9315056, WO-A-9312090, WO-A-9212969, WO-A-9118887, EP-A-0393926, EP-A-0307078, U.S. Pat. No. 5,041,442, EP-A-0266890, WO-A-9414795, EP-A-0264883, EP-A-0033094, EP-A-0259174, EP-A-0330485, WO-A-8900570, EP-A-0368158, WO-A-9117164, WO-A-9206979, WO-A-9312090, WO-A-9308190, WO-A-9418199, DE-A 3011490, U.S. Pat. No. 4,464,372, EP-A-0068378 and WO-A-9424130.

EP 0841904 B1 describes an oral pharmaceutical composition comprising reversible proton pump inhibitors in combination with antimicrobial active ingredients for the treatment of a disease caused by helicobacter. At least part of the reversible proton pump inhibitor is in extended release form.

WO-A-95/27714 is related to substituted tricyclic imidazo[1,2-a]pyridines which reversibly inhibit exogenously or endogenously stimulated gastric acid secretion. On page 38 an example for a tablet formulation is disclosed.

WO-A-0245693 discloses new preparations for an active ingredient, wherein the active ingredient is present essentially uniformly dispersed in an excipient matrix composed of one or more excipients selected from the group of fatty alcohol, triglyceride, partial glyceride and fatty acid ester. It is mentioned that the matrix is inter alia suitable for active ingredients from the class of substances known as reversible propton pump inhibitors or APAs (acid pump antagonists). Rapidly disintegrating tablets based on these preparations are mentioned.

### DESCRIPTION OF THE INVENTION

It has surprisingly been found that particularly stable oral dosage forms are obtained for proton pump antagonists (APA) when the active ingredient is stabilized in the dosage form by basic excipients. Further providing proton pump antagonists in the form of an extended release dosage form may improve the therapeutic effect of the proton pump antagonist in the treatment or prevention of gastrointestinal disorder. In particular a longer elimination of pain may be observed in the therapy of diseases derived from increased grastic acid secretion.

One aspect of the invention is therefore a stable oral dosage form for reversible proton pump inhibitors comprising an effective amount of a proton pump antagonist (APA) together with excipients, where the proton pump antagonist is stabilized in the dosage form by one or more basic excipients and which dosage form is an extended release dosage form.

Irreversible proton pump inhibitors (H⁺/K⁺-ATPase inhibitors, PPIs) are according to the invention substances which are able to bind covalently, and thus irreversibly, to the enzyme responsible for acid secretion in the stomach, H⁺/K⁺-ATPase [description of the possible mechanism of action for example in Wurst et al., The Yale Journal of Biology and Medicine 69, 3, 1996, 233-243]. By this are meant in particular pyridin-2-yl-methylsulphinyl-1H-benzimidazoles as disclosed for example in EP-A-0 005 129, EP-A-0 166 287, EP-A-0 174 726 and EP-A-0 268 956. Examples which may be mentioned are 5-methoxy-2-[(4-methoxy-3,5-dimethyl-2-pyridinyl)methylsulphinyl]-1H-benzimidazole (INN: omeprazole), 5-difluoromethoxy-2-[(3,4-dimethoxy-2-pyridinyl)methylsulphinyl]-1H-benzimidazole (INN: pantoprazole), 2-[3-methyl-4-(2,2,2-trifluoroethoxy)-2-pyridinyl)methylsulphinyl]-1H-benzimidazole (INN: lansoprazole) and 2-{[4-(3-methoxypropoxy)-3-methylpyridin-2-yl]methylsulphinyl}-1H-benzimidazole (INN: rabeprazole).

Proton pump antagonists, also called according to the invention reversible proton pump inhibitors or APA (acid pump antagonists), are for the purposes of the present invention those active ingredients able to bind reversibly to the enzyme responsible for gastric acid secretion H⁺/K⁺-ATPase [description of the possible mechanism of action of the APAs for example in Wurst et al, The Yale Journal of Biology and Medicine 69, 3, 1996, 233-243]. The term proton pump antagonists includes according to the invention not only the active ingredient as such but also the pharmacologically acceptable salts and solvates (especially hydrates) etc. Examples of proton pump antagonists are mentioned in the following documents:

EP 33094, EP 204285, EP 228006, EP 233760, EP 259174, EP 266326, EP 266890, EP 270091, EP 307078, EP 308917, EP 330485, U.S. Pat. No. 4,728,658, U.S. Pat. No. 5,362,743, WO 9212969, WO 9414795, WO 9418199, WO 9429274, WO 9510518, WO 9527714, WO 9603405, WO 9604251, WO 9605177, WO 9703074, WO 9703076, WO 9747603, WO 9837080, WO 9842707, WO 9843968, WO 9854188, WO 9909029, WO 9928322, WO 9950237, WO 9951584, WO 9955705, WO 9955706, WO 0001696, WO 0010999, WO 0011000, WO 0017200, WO 0026217, WO 0029403, WO 0063211, WO 0077003, WO 0158901, WO 0172754, WO 0172755, WO 0172756, WO 0172757, WO 02034749, WO 02060440, WO 02060441 and WO 02060442.

Examples of proton pump antagonists which may be mentioned by means of their INNs or their code designation are the compounds: AG-2000 (EP 233760), AU-461 (WO 9909029), BY112 (WO 9842707), soraprazan (BY359) (WO 0017200), CP-113411 (U.S. Pat. No. 5,362,743), DBM-819 (WO 0001696), KR-60436 (WO 9909029), pumaprazole (WO 9418199), SKF-96067 (EP 259174), SKF-96356 (EP 307078), SKF-97574 (EP 330485), T-330 (EP 270091), T-776 (EP 270091), WY-27198 (US 4728658), YH-1885 (WO 9605177), YJA-20379-8 (WO 9703074), YM-19020 (EP 266890) and 2,3-dimethyl-8-(2-ethyl-6-methylbenzylamino)imidazo[1,2-a]pyridine-6-carboxamide (WO 02060440).

Particularly worthy of mention in this connection are the compounds AU-461, soraprazan (BYK61359), DBM-819, KR-60436, T-330, YH-1885, YJA-20379-8 and 2,3-dimethyl-8-(2-ethyl-6-methylbenzylamino)imidazo[1,2-a]pyridine-6-carboxamide.

A group of APAs which is of particular interest according to the invention is described and claimed in the patent applications WO 9842707, WO 9854188, WO 0017200, WO 0026217, WO 0063211, WO 0172754, WO 0172755, WO 0172756, WO 0172757, WO 02034749, W003014120, WO03016310, WO03014123, WO03068774 and WO03091253.

Examples of APAs which may be mentioned in connection with the invention are the following compounds:

(7S,8R,9R)-2,3-dimethyl-7,8-dihydroxy-9-phenyl-7,8,9,10-tetrahydroimidazo[1,2-h][1,7]naphthyridine,

(7R,8R,9R)-3-hydroxymethyl-7,8-dihydroxy-2-methyl-9-phenyl-7,8,9,10-tetrahydroimidazo[1,2-h][1,7]naphthyridine,

(7S,8R,9R)-7,8-isopropylidenedioxy-2,3-dimethyl-9-phenyl-7,8,9,10-tetrahydroimidazo[1,2-h][1,7]naphthyridine,

7,8-dihydroxy-9-phenyl-2,3-dimethyl-7H-8,9-dihydropyrano[2,3-c]imidazo[1,2-a]pyridine,

(7R,8R,9R)-2,3-dimethyl-8-hydroxy-7-methoxy-9-phenyl-7,8,9,10-tetrahydroimidazo[1,2-h][1,7]naphthyridine,

(7S,8S,9S)-2,3-dimethyl-8-hydroxy-7-methoxy-9-phenyl-7,8,9,10-tetrahydroimidazo[1,2-h][1,7]naphthyridine,

(7S,8R,9R)-2,3-dimethyl-8-hydroxy-7-methoxy-9-phenyl-7,8,9,10-tetrahydroimidazo[1,2-h][1,7]naphthyridine,

(7R,8S,9S)-2,3-dimethyl-8-hydroxy-7-methoxy-9-phenyl-7,8,9,10-tetrahydroimidazo[1,2-h][1,7]naphthyridine,

(7R,8R,9R)-2,3-dimethyl-7-ethoxy-8-hydroxy-9-phenyl-7,8,9,10-tetrahydroimidazo[1,2-h][1,7]naphthyridine,

(7S,8R,9R)-2,3-dimethyl-7-ethoxy-8-hydroxy-9-phenyl-7,8,9,10-tetrahydroimidazo[1,2-h][1,7]naphthyridine,

(7R,8R,9R)-2,3-dimethyl-8-hydroxy-7-(2-methoxyethoxy)-9-phenyl-7,8,9,10-tetrahydroimidazo[1,2-h][1,7]naphthyridine,

(7S,8S,9S)-2,3-dimethyl-8-hydroxy-7-(2-methoxyethoxy)-9-phenyl-7,8,9,10-tetrahydroimidazo[1,2-h][1,7]naphthyridine,

(7S,8R,9R)-2,3-dimethyl-8-hydroxy-7-(2-methoxyethoxy)-9-phenyl-7,8,9,10-tetrahydroimidazo[1,2-h][1,7]naphthyridine,

(7R,8S,9S)-2,3-dimethyl-8-hydroxy-7-(2-methoxyethoxy)-9-phenyl-7,8,9,10-tetrahydroimidazo[1,2-h][1,7]naphthyridine,

(7S,8R,9R)-2,3-dimethyl-8-hydroxy-9-phenyl-7-(2-propoxy)-7,8,9,10-tetrahydroimidazo[1,2-h][1,7]naphthyridine,

(7R,8R,9R)-2,3-dimethyl-7,8-dimethoxy-9-phenyl-7,8,9,10-tetrahydroimidazo[1,2-h][1,7]naphthyridine,

(7R,8R,9R)-2,3-dimethyl-8-hydroxy-7-(2-methylthioethyloxy)-9-phenyl-7,8,9,10-tetrahydroimidazo[1,2-h][1,7]naphthyridine,

(7S,8R,9R)-2,3-dimethyl-8-hydroxy-7-(2-methylthioethyloxy)-9-phenyl-7,8,9,10-tetrahydroimidazo[1,2-h][1,7]naphthyridine,

(7R,8R,9R)-2,3-dimethyl-8-hydroxy-7-(2-methylsulphinylethoxy)-9-phenyl-7,8,9,10-tetrahydroimidazo[1,2-h][1,7]naphthyridine,

(7S,8R,9R)-2,3-dimethyl-8-hydroxy-7-(2-methylsulphinylethoxy)-9-phenyl-7,8,9,10-tetrahydroimidazo[1,2-h][1,7]naphthyridine,

(7R,8R,9R)-2,3-dimethyl-8-hydroxy-7-(ethylthio)-9-phenyl-7,8,9,10-tetrahydroimidazo[1,2-h][1,7]naphthyridine,

(7S,8R,9R)-2,3-dimethyl-8-hydroxy-7-(ethylthio)-9-phenyl-7,8,9,10-tetrahydroimidazo[1,2-h][1,7]naphthyridine,

(7R,8R,9R)-2,3-dimethyl-8-hydroxy-7-(2,2,2-trifluoroethoxy)-9-phenyl-7,8,9,10-tetrahydroimidazo[1,2-h][1,7]naphthyridine,

(7S,8R,9R)-2,3-dimethyl-8-hydroxy-7-(2,2,2-trifluoroethoxy)-9-phenyl-7,8,9,10-tetrahydroimidazol[1,2-h][1,7]naphthyridine,

(7S,8R,9R)-8-acetoxy-7-(2-methoxyethoxy)-2,3-dimethyl-9-phenyl-7,8,9, 10-tetrahydroimidazo[1,2-h][1,7]naphthyridine,

(7R,8R,9R)-8-acetoxy-7-(2-methoxyethoxy)-2,3-dimethyl-9-phenyl-7,8,9,10-tetrahydroimidazo[1,2-h][1,7]naphthyridine,

(7R,8R,9R)-8-acetoxy-7-methoxy-2,3-dimethyl-9-phenyl-7,8,9, 1 0-tetrahydroimidazo[1,2-h][1,7]naphthyridine,

(7R,8R,9R)-8-acetoxy-7-ethoxy-2,3-dimethyl-9-phenyl-7,8,9,10-tetrahydroimidazo[1,2-h][1,7]naphthyridine,

(7R,8R,9R)-7-(2-methoxyethoxy)-2,3-dimethyl-9-phenyl-8-propionyloxy-7,8,9,10-tetrahydroimidazo[1,2-h][1,7]naphthyridine,

(7R,8R,9R)-8-benzoyloxy-7-(2-methoxyethoxy)-2,3-dimethyl-9-phenyl-7,8,9,10-tetrahydroimidazo[1,2-h][1,7]naphthyridine,

(7S,8R,9R)-8-benzoyloxy-7-(2-methoxyethoxy)-2,3-dimethyl-9-phenyl-7,8,9,10-tetrahydroimidazo[1,2-h][1,7]naphthyridine,

(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,

(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,

(7R,8R,9R)-8-benzoyloxy-7-methoxy-2,3-dimethyl-9-phenyl-7,8,9,10-tetrahydroimidazo[1,2-h][1,7]naphthyridine,

(7S,8R,9R)-8-benzoyloxy-7-methoxy-2,3-dimethyl-9-phenyl-7,8,9,10-tetrahydroimidazo[1,2-h][1,7]naphthyridine,

(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,

(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,

(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,

(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,

(7S,8R,9R)-7-methoxy-2,3-dimethyl-8-(3-nitrobenzoyloxy)-9-phenyl-7,8,9,10-tetrahydroimidazo[1,2-h][1,7]naphthyridine,

(7R,8R,9R)-7-methoxy-2,3-dimethyl-8-(3-nitrobenzoyloxy)-9-phenyl-7,8,9,10-tetrahydroimidazo[1,2-h][1,7]naphthyridine,

(7S,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,

(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,

(7R,8R,9R)-7-(2-methoxyethoxy)-2,3-dimethyl-8-(N,N-dimethylaminomethylcarbonyloxy)-9-phenyl-7,8-9,10-tetrahydroimidazo[1,2-h][1,7]naphthyridine,

(7S,8R,9R)-7-(2-Methoxyethoxy)-2,3-dimethyl-8-(N,N-dimethylaminomethylcarbonyloxy)-9-phenyl-7,8-9,10-tetrahydroimidazo[1,2-h][1,7]naphthyridine,

(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,

(7R,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,

(7R,8R,9R)-8-ethylaminocarbonyloxy-7-(2-methoxyethoxy)-2,3-dimethyl-9-phenyl-7,8,9,10-tetrahydroimidazo[1,2-h][1,7]naphthyridine,

(7R,8R,9R)-8-benzoyloxy-2,3-dimethyl-7-(2-methoxyethoxy)-9-phenyl-7H-8,9-dihydropyrano[2,3-c]imidazo[1,2-a]pyridine,

(7S,8R,9R)-8-benzoyloxy-2,3-dimethyl-7-(2-methoxyethoxy)-9-phenyl-7H-8,9-dihydropyrano[2,3-c]imidazo[1,2-a]pyridine,

(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,

(7S,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,

(7S,8R,9R)-2,3-dimethyl-7-methoxy-8-methoxyacetyloxy-9-phenyl-7,8,9,10-tetrahydroimidazo[1,2-h][1,7]naphthyridine,

(7R,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,

(7S,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,

(7R,8R,9R)-7-methoxy-8-methoxycarbonyloxy-2,3-dimethyl-9-phenyl-7,8,9,10-tetrahydroimidazo[1,2-h][1,7]naphthyridine,

(7S,8R,9R)-7-methoxy-8-methoxycarbonyloxy-2,3-dimethyl-9-phenyl-7,8,9,10-tetrahydroimidazo[1,2-h][1,7]naphthyridine,

(7R,8R,9R)-2,3-dimethyl-8-formyloxy-7-methoxy-9-phenyl-7,8,9,10-tetrahydroimidazo[1,2-h][1,7]naphthyridine,

(7S,8R,9R)-2,3-dimethyl-8-formyloxy-7-methoxy-9-phenyl-7,8,9,10-tetrahydroimidazo[1,2-h][1,7]naphthyridine,

(7R,8R,9R)-8-benzoyloxy-2,3-dimethyl-7-methoxy-9-phenyl-7,8,9,10-tetrahydroimidazo[1,2-h][1,7]naphthyridine,

(7R,8S,9R)-2,3,8-trimethyl-7,8-dihydroxy-9-phenyl-7,8,9,10-tetrahydroimidazo[1,2-h][1,7]naphthyridine,

(7S,8S,9R)-2,3-dimethyl-8-benzyl-7,8-dihydroxy-9-phenyl-7,8,9,10-tetrahydroimidazo[1,2-h][1,7]naphthyridine,

(7R,8S,9R)-2,3,8-trimethyl-7,8-0,0-isopropylidene-9-phenyl-7,8,9,10-tetrahydroimidazo[1,2-h][1,7]naphthyridine,

(7S,8S,9R)-2,3,8-trimethyl-7-(2-methoxyethoxy)-8-hydroxy-9-phenyl-7,8,9,10-tetrahydroimidazo[1,2-h][1,7]naphthyridine,

(7S,8S,9R)-2,3,8-trimethyl-7-methoxy-8-hydroxy-9-phenyl-7,8,9,10-tetrahydroimidazo[1,2-h][1,7]naphthyridine,

(7R,8R,9R)-2,3,7-trimethyl-7,8-dihydroxy-9-phenyl-7,8,9,10-tetrahydroimidazo[1,2-h][1,7]naphthyridine,

(7R,8R,9R)-2,3,7-trimethyl-7,8-[1,3]dioxolo-9-phenyl-7,8,9,10-tetrahydroimidazo[1,2-h][1,7]naphthyridine,

(8S,9R)-2,3-dimethyl-8-hydroxy-7-methylidene-9-phenyl-7,8,9,10-tetrahydroimidazo[1,2-h]1,7]naphthyridine,

(7S,8R,9R)-2,3,7-trimethyl-7,8-dihydroxy-9-phenyl-7H-8,9-dihydropyrano[2,3-c]imidazo[1,2-a]pyridine,

(7R,8R,9R)-2,3,7-trimethyl-7,8-dihydroxy-9-phenyl-7H-8,9-dihydropyrano[2,3-c]imidazo[1,2-a]pyridine,

(7S,8R,9R)-2,3-dimethyl-7,8-dihydroxy-7,9-diphenyl-7H-8,9-dihydropyrano[2,3-c]imidazo[1,2-a]pyridine,

(7S,8R,9R)-2,3-dimethyl-7-(2′,2′-dimethylvinyl)-7,8-dihydroxy-9-phenyl-7H-8,9-dihydropyrano[2,3-c]imidazo[1,2-a]pyridine,

(7R,8R,9R)-2,3-dimethyl-7,8-O-isopropylidene-9-phenyl-7-vinyl-7H-8,9-dihydropyrano[2,3-c]imidazo[1,2-a]pyridine,

(7R,8R,9R)-2,3-dimethyl-8-hydroxy-7-(2-methoxyethoxy)-9-phenyl-7H-8,9-dihydropyrano[2,3-c]imidazo[1,2-a]pyridine,

(7S,8R,9R)-2,3-dimethyl-8-hydroxy-7-(2-methoxyethoxy)-9-phenyl-7H-8,9-dihydropyrano[2,3-c]imidazo[1,2-a]pyridine,

(7R,8R,9R)-2,3-dimethyl-8-hydroxy-7-ethoxy-9-phenyl-7H-8,9-dihydropyrano[2,3-c]imidazol[1,2-a]pyridine,

(7S,8R,9R)-2,3-dimethyl-8-hydroxy-7-ethoxy-9-phenyl-7H-8,9-dihydropyrano[2,3-c]imidazol[1,2-a]pyridine,

(7R,8R,9R)-2,3-dimethyl-8-hydroxy-7-(2-methoxypropoxy)-9-phenyl-7H-8,9-dihydropyrano[2,3-c]imidazo[1,2-a]pyridine,

(7S,8R,9R)-2,3-dimethyl-8-hydroxy-7-(2-methoxypropoxy)-9-phenyl-7H-8,9-dihydropyrano[2,3-c]imidazo[1,2-a]pyridine,

(7R,8R,9R)-2,3-dimethyl-8-hydroxy-7-(2-propoxy)-9-phenyl-7H-8,9-dihydropyrano[2,3-c]imidazol[1,2-a]pyridine,

(7S,8R,9R)-2,3-dimethyl-8-hydroxy-7-(2-propoxy)-9-phenyl-7H-8,9-dihydropyrano[2,3-c]imidazol[1,2-a]pyridine,

(7R,8R,9R)-2,3-dimethyl-8-hydroxy-7-butoxy-9-phenyl-7H-8,9-dihydropyrano[2,3-c]imidazol[1,2-a]pyridine,

(7S,8R,9R)-2,3-dimethyl-8-hydroxy-7-butoxy-9-phenyl-7H-8,9-dihydropyrano[2,3-c]imidazol[1,2-a]pyridine,

(7S,8R,9R)-7,8-dihydroxy-6-methoxymethyl-2,3-dimethyl-9-phenyl-7,8,9,10-tetrahydroimidazo[1,2-h][1,7]naphthyridine,

(7R,8R,9R)-7,8-dihydroxy-6-methoxymethyl-2,3-dimethyl-9-phenyl-7,8,9,10-tetrahydroimidazo[1,2-h][1,7]naphthyridine,

(7S,8R,9R)-8-hydroxy-7-methoxy-6-methoxymethyl-2,3-dimethyl-9-phenyl-7,8,9,10-tetrahydroimidazo[1,2-h][1,7]naphthyridine,

(7R,8R,9R)-8-hydroxy-7-methoxy-6-methoxymethyl-2,3-dimethyl-9-phenyl-7,8,9,10-tetrahydroimidazo[1,2-h][1,7]naphthyridine,

(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,

(7S,8R,9R)-8-Hydroxy-7-(2-methoxyethoxy)-6-methoxymethyl-2,3-dimethyl-9-phenyl-7,8,9,10-tetrahydroimidazo[1,2-h][1,7]naphthyridine,

(7R,8R,9R)-8-hydroxy-7-ethoxy-6-methoxymethyl-2,3-dimethyl-9-phenyl-7,8,9,10-tetrahydro-imidazo[1,2-h][1,7]naphthyridine,

(7S,8R,9R)-8-hydroxy-7-ethoxy-6-methoxymethyl-2,3-dimethyl-9-phenyl-7,8,9,10-tetrahydro-imidazo[1,2-h][1,7]naphthyridine,

7,8-dihydroxy-2,3-dimethyl-9-(3-thienyl)-7,8,9,10-tetrahydroimidazo[1,2-h][1,7]naphthyridine,

7-hydroxy-2,3-dimethyl-9-(3-thienyl)-7,8,9, 1 0-tetrahydroimidazo[1,2-h][1,7]naphthyridine,

9-(3-furyl)-7-hydroxy-2,3-dimethyl-7,8,9,10-tetrahydroimidazo[1,2-h][1,7]naphthyridine,

(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,

(7S,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,

(7R,8R,9R)-7,8-dihydroxy-2-methyl-9-phenyl-7,8,9,10-tetrahydroimidazo[1,2-h][1,7]naphthyridine,

(7S,8R,9R)-8-hydroxy-2-methyl-7-(2-methoxyethoxy)-9-phenyl-7,8,9,10-tetrahydroimidazo[1,2-h][1,7]naphthyridine,

(7R,8R,9R)-8-hydroxy-2-methyl-7-(2-methoxyethoxy)-9-phenyl-7,8,9,10-tetrahydroimidazo[1,2-h][1,7]naphthyridine,

(7R,8R,9R)-3-bromo-8-hydroxy-7-(2-methoxyethoxy)-2-methyl-9-phenyl-7,8,9,10-tetrahydroimidazo[1,2-h][1,7]naphthyridine,

(7R,8R,9R)-3-chloro-8-hydroxy-7-(2-methoxyethoxy)-2-methyl-9-phenyl-7,8,9,10-tetrahydroimidazo[1,2-h][1,7]naphthyridine,

(7R,8R,9R)-3-bromo-7-hydroxy-8-(2-methoxyethoxy)-2-methyl-9-phenyl-7,8,9,10-tetrahydroimidazo[1,2-h][1,7]naphthyridine,

(7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine,

(7R,8R,9R)-8-hydroxy-7-(2-methoxyethoxy)-2-methyl-9-phenyl-7H-8,9-dihydro-pyrano[2,3-c]imidazo[1,2-a]pyridine,

(7R,8R,9R)-7,8-dihydroxy-2-methyl-9-phenyl-7H-8,9-dihydropyrano[2,3-c]imidazo[1,2-a]pyridine,

(7S,8R,9R)-7,8-dihydroxy-2-methyl-9-phenyl-7,8,9,10-tetrahydroimidazo[1,2-h][1,7]naphthyridine,

(7R,8R,9R)-8-hydroxy-7-methoxy-2-methyl-9-phenyl-7,8,9,10-tetrahydroimidazo[1,2-h][1,7]naphthyridine,

(7S,8R,9R)-8-hydroxy-7-methoxy-2-methyl-9-phenyl-7,8,9,10-tetrahydroimidazo[1,2-h][1,7]naphthyridine,

(7R,8R,9R)-3-hydroxymethyl-8-hydroxy-7-(2-methoxyethoxy)-2-methyl-9-phenyl-7,8,9,10-tetrahydroimidazo[1,2-h][1,7]naphthyridine,

(7R,8R,9R)-3-hydroxymethyl-8-hydroxy-7-(2-hydroxyethoxy)-2-methyl-9-phenyl-7,8,9,10-tetrahydroimidazo[1,2-h][1,7]naphthyridine,

(7R,8R,9R)-2,3-dimethyl-8-hydroxy-7-(2-hydroxyethoxy)-9-phenyl-7,8,9,10-tetrahydroimidazol[1,2-h][1,7]naphthyridine,

(7R,8R,9R)-3,9-diphenyl-8-hydroxy-7-(2-methoxyethoxy)-2-methyl-7,8,9,10-tetrahydroimidazo[1,2-h][1,7]naphthyridine,

(7R,8R,9R)-7,8-dihydroxy-2-methoxymethyl-3-methyl-9-phenyl-7,8,9,10-tetrahydroimidazo[1,2-h][1,7]naphthyridine,

(7S,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,

(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,

(7S,8R,9R)-7-ethoxy-8-hydroxy-2-methoxymethyl-3-methyl-9-phenyl-7,8,9,10-tetrahydroimidazo[1,2-h][1,7]naphthyridine,

(7R,8R,9R)-7-ethoxy-8-hydroxy-2-methoxymethyl-3-methyl-9-phenyl-7,8,9,10-tetrahydroimidazo[1,2-h][1,7]naphthyridine,

(8S)-2,3-Dimethyl-8-phenyl-7,8-dihydro-6H-9oxa-1,3a-diaza-cyclopenta[a]naphthalene-5-carboxylic acid dimethylamid,

8-[(1S,2S)-2,3-dihydro-2-hydroxy-1-indenyloxy-6-(N,N-dimethylaminocarbonyl)-2,3-dimethyl-imidazo[1,2-a]pyridine,

6-(N,N-Dimethylaminocarbonyl)-4-(2,6-dimethyl-benzylamino)-1,2-dimethyl-1H-benzimidazole,

and the pharmacologically suitable salts of these compounds.

An example of a preferred proton pump antagonist which may be mentioned is the compound (7R,8R,9R)-2,3-dimethyl-8-hydroxy-7-(2-methoxyethoxy)-9-phenyl-7,8,9,10-tetrahydroimidazo[1,2-h][1,7]naphthyridine (INN: soraprazan).

The proton pump antagonists may in this connection be present as such or in the form of their salts and/or solvates (e.g. hydrates) etc. Most reversible proton pump inhibitors are basic compounds. Particularly suitable salts are all acid addition salts. Particular mention may be made of the pharmacologically acceptable salts of the inorganic and organic acids normally used in pharmaceutical technology. Suitable as such are water-soluble and water-insoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in the preparation of the salts, depending on whether the acid is mono- or polybasic and on which salt is desired—in the equimolar ratio of amounts or one differing therefrom.

Extended release (hereinafter also referred to as controlled release, retarding release, slow release, prolonged action and sustained release) dosage form in connection with the present invention relates to a dosage form that does not release active drug substance immediately after oral dosing. In a preferred embodiment extended release dosage form relates to a dosage form that allows a reduction in dosage frequency as compared to a immediate drug-releasing, conventional solid dosage form. In a preferred embodiment the dosage form according to the invention is a sustained release dosage form which shows a dissolution (release of active ingredient) of less than 90% in at least three hours in 0.1 N hydrochloric acid.

A dosage form means, in particular, those medicinal dosage forms in which slowing or extending of proton pump antagonist release is achieved with as few problems as possible. These include, in particular, tablets, coated tablets or pellets, and microtablets in capsules, with the dosage form advantageously being designed so that the active ingredient is released, or made available effectively for the body, in such a way that an optimal active-ingredient profile (and thus action profile) is achieved.

For extending release, various types and degrees of retarding release may be used to ensure a proton pump antagonist plasma level which persists as long as possible and is sufficient for raising pH.

The details of how to achieve slowing release are familiar to the skilled worker on the basis of his expert knowledge. To this end the dosage form according to the invention contains one or more excipients which on oral intake of the dosage form bring about sustained release of the proton pump antagonist. The skilled worker is likewise familiar with suitable ancillary substances and vehicles for the required dosage forms (pharmaceutical formulations). Besides solvents, tablet ancillary substances and other active ingredient excipients it is possible to use, for example, tablet-coating compositions, plasticizers, antioxidants, preservatives, dyes, etc. Where incompatibilities between the active ingredient and ancillary substances are to be expected, suitable separating layers must be provided where appropriate.

The dosage forms according to the invention are distinguished by sustained release of active ingredient and an optimal action profile (e.g. a constant blood level) in the therapy of diseases derived from increased gastric acid secretion. There is furthermore observed to be an improved stability of the proton pump antagonists in dosage forms according to the invention containing a basic excipient.

Basic excipients which are suitable according to the invention and which can be employed in the dosage forms according to the invention to stabilize the proton pump antagonists are substances which have a basic reaction and are pharmacologically acceptable and able to stabilize the proton pump antagonists in the dosage form. These are, in particular, compounds selected from the group of pharmacologically acceptable alkali metal, alkaline earth metal or earth metal salts of weak acids, pharmacologically suitable hydroxides and oxides of alkaline earth and earth metals or else pharmacologically acceptable basic buffer systems. Examples which may be mentioned are sodium carbonate, calcium carbonate, magnesium carbonates, magnesium oxide, magnesium hydroxide, magnesium metasilicate aluminate, magnesium silicates, magnesium aluminate, hydrotalcite (synthetic), aluminium magnesium hydroxide, and calcium hydroxide, basic salts of amino acids, sodium hydroxide, trihydroxymethylaminomethane, trisodium citrate, disodium hydrogen phosphate and trisodium phosphate or mixtures thereof.

Preference is given according to the invention to sodium carbonate, disodium hydrogen phosphate, trisodium phosphate and buffer systems composed of disodium hydrogen phosphate with sodium hydroxide.

The basic excipient is preferably thoroughly mixed in finely divided form with the active ingredient and, where appropriate, other excipients or carriers so that there is intensive (direct) contact between basic excipient and the active ingredient. A further possibility is also to employ excipient granules impregnated with a basic buffer system.

The basic excipient is preferably added in an amount such that when 100 mg of mixtures of the active ingredient with the desired excipients are dissolved in 50 ml of purified water the basicity reaches not less than pH 7, preferably a basicity of pH 8 to pH 11.5, particularly preferably of pH 8 to pH 11,0 and very particularly preferably of pH 8.5 to 10.5. Depending on the nature of the basic excipient, the content can therefore be for example from 0.1 to 30% by weight (in per cent by weight based on the finished dosage form). In a preferred embodiment the content of the basic excipient is below 20% by weight, particularly preferable below 15% by weight and in particular below 10% by weight (in per cent by weight based on the finished dosage form).

Excipients for sustained release which may be mentioned in connection with the invention are polymers for coatings. Suitable for the film coating in the case of coated dosage forms according to the invention (such as, for example, coated tablets and pellets) are substances suitable for film coating. Examples which may be mentioned are cellulose esters such as cellulose acetate phtalate (CAP) and ethylcellulose, polyvinyl acetate, acrylates and methacrylates, acrylate dispersions and shellac, to which plasticizers (such as, for example, propylene glycol, polyethylene glycol, trisodium citrate) and/or further additives and excipients (e.g. buffers, bases such as, preferably, aluminium hydroxide or pigments) can also be added, if desired. In the case of film coatings, the concentration of dry polymer substance (in % by weight based on the final dosage form) is from 1 to 20% by weight, preferably from 3 to 10% by weight. Alternatively, ion exchange resins with pH-controlled release of active substances such as polystyrol or methycrylic resins with exchangeable ions and acid or basic groups, are possible. Suitable mediums for applying the coating to for example the tablet core can be produced with water and/or organic solvents. In addition, coagulation or coacervation processes might be used for manufacture of coated dosage forms.

Further excipients suitable for sustaining release of the active ingredient, which may be mentioned in connection with the invention are matrix forming excipients. Suitable for a matrix tablet according to the invention are excipients like lipids (e.g. stearyl alcohol, cetostearyl alcohol, cetyl alcohol, stearic acid, cottonseed oil, hydrogenated castor oil, Precirol®), plastic materials with thermoplastic properties (e.g. polyvinylacetate, polyvinylchloride, polyethylene) and swelling materials, forming a gel layer, which delayes the diffusion of active drug substances (e.g. methylcellulose and hydroxypropylmethylcellulose). In the case of matrix tablets, the content (in % by weight based on the finished dosage form) is from 1 to 30% by weight, preferably from 5 to 20% by weight.

Further excipients which can be used in the dosage forms according to the invention are, for example, excipients which influence the disintegration time of the core by effecting an osmotic pressure into the dosage form on oral intake of the dosage form. These preferably comprise one or more substances selected from the group of fillers or carriers and disintegrants. It is furthermore possible for one or more excipients from the group of binders, lubricants, colouring agents, aromas, flavourings and surface-active substances to be present.

Fillers or carriers suitable according to the invention are, in particular, fillers such as calcium carbonate (e.g. MagGran® CC or Destab® 95) and sodium carbonate, sugar alcohols such as mannitol (e.g. Perlitol® or Parteck® M), sorbitol (e.g. Karion®), xylitol or maltitol, starches such as corn starch, potato starch and wheat starch, microcrystalline cellulose, saccharides such as glucose, lactose, levulose, sucrose and dextrose. Microcrystalline cellulose and/or mannitol are particularly preferred.

The content (in per cent by weight based on the finished dosage form) of filler in the dosage form according to the invention is advantageously from 1 to 99% by weight. The content of filler is preferably from 30 to 95% by weight, and the content is particularly preferably from 60 to 90% by weight.

Disintegrants suitable according to the invention are, in particular, insoluble polyvinylpyrrolidone (insoluble PVP, crosspovidone), sodium carboxymethyl starch, sodium carboxymethylcellulose, alginic acid, and starches able to fulfil the function of a disintegrant (e.g. Starch 1500).

The content (in per cent by weight based on the dosage form according to the invention) of disintegrant in the dosage form according to the invention can usually be from 0.5 to 30% by weight. The content of disintegrant is preferably from 1 to 15% by weight. The content of disintegrant is particularly preferably from 1 to 5% by weight.

Suitable lubricants which may be mentioned are sodium stearyl fumarate, magnesium stearate, calcium stearate, stearic acid, talc and colloidal silica (Aerosil).

The content (in per cent by weight based on the finished dosage form) of lubricant in the sustained release dosage form according to the invention is usually from 0.1 to 5% by weight. The content of lubricant is preferably from 0.2 to 3% by weight. The content of lubricant is particularly preferably from 0.5 to 2% by weight.

Binders suitable according to the invention are polyvinylpyrrolidone (PVP, Polyvidon® K25, Polyvidon® K90) or mixtures of PVP with polyvinyl acetate (e.g. Kollidon® 64), gelatin, corn starch paste, preswollen starches (Starch® 1500, Uni-Pure® WG220), hydroxypropylmethylcellulose (HPMC) or hydroxypropylcellulose (L-HPC).

The content (in per cent by weight based on the finished dosage form according to the invention) of binder can be up to 10% by weight, and it can preferably be up to 5% by weight.

Suitable surface-active substances which may be mentioned are sodium lauryl sulfate or Tween® 20, Tween® 60 or Tween® 80.

The dosage form according to the invention particularly preferably contains a mixture of at least one basic excipient, one filler or carrier and one lubricant. In dependence of the release system, a disintegrant can be used.

A dosage form which may be mentioned as preferred in this connection is one containing microcrystalline cellulose as filler or carrier and sodium carbonate as basic excipient and a lubricant. In another embodiment, the dosage form according to the invention contains a mixture of at least one basic excipient, one filler or carrier, one binder and one lubricant. A dosage form which may be mentioned as preferred in this connection is one containing a mixture which contains mannitol and microcrystalline cellulose as filler or carrier, sodium carbonate as basic excipient and a binder. Another dosage form which may be mentioned as preferred in this connection is one containing a mixture which contains microcrystalline cellulose, sodium carbonate, sodium carboxymethyl starch and magnesium stearate.

It is also possible if desired for one or more flavourings (e.g. aromas or sweeteners) to be present in the dosage form according to the invention. It is possible thereby for example to achieve an improvement in taste. These substances are added in the usual amounts.

In the case of dosage forms containing photosensitive reversible proton pump inhibitors it is preferred for a coloured film coating to be applied to the dosage forms according to the invention or for dyes to be incorporated directly into the dosage forms. Suitable colouring agents are for example, iron oxides, Indigocarmin E132 or titanium dioxide.

Besides filler and binder, other ancillary substances, in particular lubricants and nonstick agents, and tablet disintegrants, are used in the manufacture of the tablet cores. A suitable binder is, in particular, polyvinylpyrrolidone in various degrees of polymerization. Examples of lubricants and nonstick agents are higher fatty acids and their alkali-metal and alkaline-earth-metal salts, such as calcium stearate. Suitable tablet disintegrants are, in particular, chemically-inert agents. Preferred tablet disintegrants include cross-linked polyvinylpyrrolidone, crosslinked sodium carboxymethylcelluloses and sodium starch glycolate.

In one embodiment the dosage form according to the invention relates to a tablet with a film coating, which film coating is customary for sustained-release compositions.

Film coatings customary for sustained-release compositions which may be mentioned are membranes made of plastics having a low swelling power in water, in which small soluble particles are embedded, or in particular those swellable plastic membranes which contain a small proportion of a suitable salt which determines the permeability of the film coating.

Plastics suitable for the construction of the membranes are those which are water-insoluble and physiologically tolerable. Plastics having a low swelling power in water are understood for the purposes of the present invention as meaning, for example, those which absorb not more than 5% by weight of water in aqueous medium. For this, cellulose ethers and cellulose esters are regarded as particularly suitable. In addition, suitable plastics are also polymers such as polyvinyl chloride. Swellable plastics which may be mentioned are, in particular, copolymers of acrylic and methacrylic acid esters.

Small soluble particles which may be mentioned are, for example, lactose crystals, which are preferably employed in micronized form. The particle size is expediently less than 20 μm, preferably less than 10 μm. The ratio of plastic to soluble particles can be varied within wide limits. A weight ratio of plastic to soluble particles of approximately 2:1 to 1:3 is preferred. A weight ratio of 4:3 to 4:5 is particularly preferred.

Salts suitable for the swellable plastic membranes which may be mentioned are, for example, ammonium salts, in particular quaternary ammonium salts. In a particular embodiment of plastic membranes, some of the ester groups of a copolymer of acrylic and methacrylic acid esters are ester groups having quaternary ammonium structures. An example of such copolymers having quaternary ammonium groups which may be mentioned is trimethylammonium methyl methacrylate chloride (e.g. Eudragit RL or Eudragit RS from Röhm).

Another example of film coatings which may be mentioned in connection with the present invention is SURELEASE®, a dispersion of ethylcellulose in water.

The release time of the proton pump antagonist can be controlled within a wide range by variation of the composition of the membrane and/or by variation of the layer thickness of the membrane. Thus, release is effected at an earlier time by lowering the layer thickness of the membrane, by increasing the proportion of soluble particles, by use of the soluble particles in a more coarse-grained form or, in the case of the swellable plastic membranes, by increasing the proportion of a suitable salt (e.g. higher proportion of quaternary ammonium groups in the copolymer of acrylic and methacrylic acid esters).

The application of the membrane to the tablet cores is carried out in a manner known per se, in particular by one of the customary spraying techniques. For this, a solution of the plastic or plastic mixture intended for the membrane is prepared in a solvent or in a solvent mixture or preferably an aqueous dispersion of the plastic or plastic mixture. The soluble, micronized particles are suspended in the solution before the spraying. If necessary, the suspension is stirred during the spraying in order to prevent settling of the suspended particles. In the case of the preferred procedure using aqueous dispersions, the salts responsible for the permeability of the plastic are already contained in the plastic itself in the form of quaternary ammonium groups. In the case of application of the membrane from an aqueous dispersion, it is also possible to work under alkaline conditions.

The membrane can contain the customary auxiliaries, such as plasticizers, wetting agents, colorants and antiadherents. Pharmacologically tolerable plasticizers such as, for example, polyethylene glycols, paraffins, glycerol or propylene glycol are suitable. Wetting agents may be necessary if the coating is to be dyed with dye lakes. Sorbitol fatty acid esters or salts of dioctylsulfosuccinic acid, for example, are suitable. Antiadherents which may be mentioned are, in particular, calcium stearate or talc.

In another embodiment according to the invention, the dosage form according the invention is a matrix tablet containing excipients suitable for sustaining release of the active ingredient. The sustained release is effected by producing a matrix tablet, for example with excipients such as hydroypropylmethylcellulose or copolymers having quaternary ammonium groups as trimethylammonium methyl methacrylate chloride (e.g. Eudragit RL or Eudragit RS from Röhm).

In a further embodiment according to the invention the dosage form according to the invention is a matrix tablet containing excipients suitable for sustaining release of the active ingredient and further containing a film coating which film coating is customary for sustained-release compositions. In an embodiment according to the invention only part of the proton pump antagonist is in extended release form and another part of the proton pump antagonist is in rapid (immediate) release form. The rapid release of part of the proton pump antagonist and retarding release of another part is optionally achieved, for example, by layered tablets or multilayer tablets, in which part of the reversible proton pump inhibitor is present in an outer coating in a form without slowing release; this is followed by another coating containing the antimicrobially-active ingredient and then the core with the reversible proton pump inhibitor whose release is slowed in a suitable manner. The dosage form according to the invention may also be combined with a conventional (i.e. immediate release) dosage form comprising the proton pump antagonist (e.g. capsule comprising immediate release pellets and pellets according to the invention, or in particular in a capsule comprising two or more tablets, of which at least one corresponds to the specification according to the invention).

In a preferred embodiment according to the invention the dosage form is comprising (7R,8R,9R)-2,3-dimethyl-8-hydroxy-7-(2-methoxyethoxy)-9-phenyl-7,8,9,10-tetrahydroimidazo[1,2-h][1,7]naphthyridine (INN soraprazan) or a pharmacologically acceptable salt and/or hydrate thereof as proton pump antagonist, sodium carbonate as basic exipient and microcrystalline cellulose, sodium carboxymethyl starch and magnesium stearate as exipients. In a further embodiment such dosage form is a film coated tablet. Particularly preferably such dosage form comprises a filmcoating suitable for sustained release of the active ingredient.

The dosage form according to the invention is produced by processes known to the skilled person, in particular by mixing the proton pump antagonists with the excipients. It is preferred in this connection for the active ingredient to be mixed thoroughly with the basic excipient. In the case of tablets, the sustained release dosage form is preferably produced by dry mixing of the excipients with the active ingredient. If desired, the active ingredient can be premixed with part of the filler or carrier. Conventional mixers such as compulsory mixers or free-fall mixers can be employed for the mixing operation. An alternative possibility is to produce granules of the ingredients of the dosage form and then compress them to tablets. The preparations obtained in this way can then be compressed on a suitable tablet press. If desired, precompaction may also take place. In the case of coated tablets, the desired film coating is then applied in conventional ways using the equipment customary for these purposes (e.g. coating pans or drum coaters). Water is preferably used as granulating and coating medium. In the case of coloured dosage forms the colouring agent is preferably dispersed homogeneously in the granules, or admixed dry, and then moistened or granulated or suspended in the dye pigment in the granulating liquid.

In the case of pellets, the sustained release dosage form is preferably produced by spraying a basified active ingredient preparation onto starter pellets or by the extruder/spheronizer process.

### EXAMPLES

The following formulation examples illustrate the invention in detail without restricting it.

### EXAMPLES

### Example 1

Film-Coated Tablets:

I. Production of the uncoated core:

| | | |
|---|---|---|
| a) | soraprazan | 10.0 mg |
| b) | sodium carbonate (anhydrous) | 5.1 mg |
| c) | microcrystalline cellulose | 137.2 mg |
| | (e.g.: Avicel PH 102) | |
| d) | microcrystalline cellulose | 7.5 mg |
| | (e.g.: Avicel PH 101) | |
| e) | sodium carboxymethyl starch | 8.5 mg |
| f) | magnesium stearate | 1.7 mg |
| | | 170.0 mg |

a) is premixed with d) in a compulsory mixer. This mixture is admixed with b), c) and e) in the compulsory mixer. Subsequently f) is admixed in a free-fall mixer. The tabletting mixture is compressed to cores in a suitable tablet press.

II. Film layer

| | | |
|---|---|---|
| g) | Surelease E7-7050 | 5.0 mg |
| | | 175.0 mg |

g) is applied to the tablet cores obtained in 1. in a suitable film-coating apparatus.

### Example 2

Film-Coated Matrix Tablets:

I. Production of the uncoated core:

| | | |
|---|---|---|
| a) | soraprazan | 10.0 mg |
| b) | trisodium phosphate | 5.1 mg |
| c) | HPMC 2208 | 20.0 mg |
| d) | microcrystalline cellulose | 82.2 mg |
| | (e.g.: Avicel PH 101) | |
| e) | mannitol | 51.0 mg |
| f) | magnesium stearate | 1.7 mg |
| | | 170.0 mg |

a)-e) are granulated, f) is added in a free-fall mixer. The tabletting mixture is compressed to cores in a suitable tablet press.

II. Film layer

| | | |
|---|---|---|
| i) | Opadry II green | 3.1 mg |
| | | 173.1 mg |

### Example 3

Film-Coated Matrix Tablets:

I. Production of the uncoated core:

| | | |
|---|---|---|
| a) | soraprazan | 20.0 mg |
| b) | sodium carbonate (anhydrous) | 10.2 mg |
| c) | Eudragit RS 30D | 20.0 mg |
| d) | microcrystalline cellulose | 103.1 mg |
| | (e.g.: Avicel PH 102) | |
| e) | microcrystalline cellulose | 15.0 mg |
| | (e.g.: Avicel PH 101) | |
| f) | magnesium stearate | 1.7 mg |
| | | 170.0 mg |

a)-e) are granulated. f) is admixed in a free-fall mixer. The tabletting mixture is compressed to cores in a suitable tablet press.

II. Film layer

| | | |
|---|---|---|
| g) | Opadry II green | 5.0 mg |
| h) | sodium carbonate (anhydrous) | 1.0 mg |
| | | 176.0 mg |

g) is applied to the tablet cores obtained in I. in a suitable film-coating apparatus.

### Example 4

Film-Coated Matrix Tablets:

I. Production of the uncoated core:

| | | |
|---|---|---|
| a) | soraprazan | 20.0 mg |
| b) | sodium carbonate (anhydrous) | 10.2 mg |
| c) | Eudragit RL 30 D | 70.0 mg |
| d) | microcrystalline cellulose | 221.4 mg |
| | (e.g.: Avicel PH 102) | |
| e) | microcrystalline cellulose | 15.0 mg |
| | (e.g.: Avicel PH 101) | |
| f) | magnesium stearate | 3.4 mg |
| | | 340.0 mg |

a)-e) are granulated. f) is admixed briefly in a free-fall mixer. The tabletting mixture is compressed to cores in a suitable tablet press.

II. Film layer

| | | |
|---|---|---|
| g) | Opadry II green | 7.5 mg |
| | | 347.5 mg |

g) is applied to the tablet cores obtained in I. in a suitable film-coating apparatus.

### Example 5

Film-Coated Tablets:

I. Production of the uncoated core:

| | | |
|---|---|---|
| a) | soraprazan | 20.0 mg |
| b) | sodium carbonate (anhydrous) | 5.1 mg |
| c) | microcrystalline cellulose | 119.7 mg |
| | (e.g.: Avicel PH 102) | |
| d) | microcrystalline cellulose | 15.0 mg |
| | (e.g.: Avicel PH 101) | |
| e) | Primojel | 8.5 mg |
| f) | magnesium stearate | 1.7 mg |
| | | 170.0 mg |

a) is premixed with d) in a compulsory mixer. This mixture is admixed with b), c) and e) in the compulsory mixer. Subsequently f) is admixed briefly in a free-fall mixer. The tabletting mixture is compressed to cores in a suitable tablet press.

II. Film layer

| | Ingredients | Coating per core |
|---|---|---|
| (g) | Eudragit RS 30 D | 4.876 mg |
| (h) | purified water | |
| (i) | propylene glycol | 0.975 mg |
| (j) | Ca stearate | 0.146 mg |
| (k) | 1 N NaOH | 0.002 mg |

| | |
|---|---|
| Total film coating | 6.000 mg |
| Weight per film | 176.0 mg |
| coated tablet: | |

The ingredients are stirred to give a dispersion which is screened before processing. The dispersion is sprayed onto the cores obtained under I in a suitable apparatus.

### Example 6

Film-Coated Tablets:

I. Production of the uncoated core:

| | | |
|---|---|---|
| a) | soraprazan | 20.0 mg |
| b) | sodium carbonate (anhydrous) | 5.1 mg |
| c) | microcrystalline cellulose | 119.7 mg |
| | (e.g.: Avicel PH 102) | |
| d) | microcrystalline cellulose | 15.0 mg |
| | (e.g.: Avicel PH 101) | |
| e) | Primojel | 8.5 mg |
| f) | magnesium stearate | 1.7 mg |
| | | 170.0 mg |

a) is premixed with d) in a compulsory mixer. This mixture is admixed with b), c) and e) in the compulsory mixer. Subsequently f) is admixed briefly in a free-fall mixer. The tabletting mixture is compressed to cores in a suitable tablet press.

II. Film layer

| Ingredients | | Coating per core |
|---|---|---|
| (g) | Eudragit RS 30 D | 2.438 mg |
| | Eudragit RL 30 D | 2.438 mg |
| (h) | purified water | |
| (i) | propylene glycol | 0.975 mg |
| (j) | Ca stearate | 0.146 mg |
| (k) | 1 N NaOH | 0.002 mg |

| | |
|---|---|
| Total film coating | 6.000 mg |
| Weight per film | 176.0 mg |
| coated tablet: | |

The ingredients are stirred to give a dispersion which is screened before processing. The dispersion is sprayed onto the cores obtained under I in a suitable apparatus.

### Example 7

Film-Coated Tablets:

I. Production of the uncoated core:

| | | |
|---|---|---|
| a) | soraprazan | 20.0 mg |
| b) | sodium carbonate (anhydrous) | 5.1 mg |
| c) | microcrystalline cellulose | 126.7 mg |
| | (e.g.: Avicel PH 102) | |
| d) | microcrystalline cellulose | 15.0 mg |
| | (e.g.: Avicel PH 101) | |
| e) | Primojel | 1.5 mg |
| f) | magnesium stearate | 1.7 mg |
| | | 170.0 mg |

a) is premixed with d) in a compulsory mixer. This mixture is admixed with b), c) and e) in the compulsory mixer. Subsequently f) is admixed briefly in a free-fall mixer. The tabletting mixture is compressed to cores in a suitable tablet press.

II. Film layer

| Ingredients | | Coating per core |
|---|---|---|
| (g) | Eudragit NE 30 D | 9.754 mg |
| (h) | purified water | |
| (i) | propylene glycol | 1.950 mg |
| (j) | Ca stearate | 0.292 mg |
| (k) | 1 N NaOH | 0.004 mg |

| | |
|---|---|
| Total film coating | 12.000 mg |
| Weight per | 182.0 mg |
| filmcoated tablet: | |

The ingredients are stirred to give a dispersion which is screened before processing. The dispersion is sprayed onto the cores obtained under I in a suitable apparatus.

### Example 8

Film-Coated Tablets:

I. Production of the uncoated core:

| | | |
|---|---|---|
| a) | soraprazan | 10.0 mg |
| b) | sodium carbonate (anhydrous) | 5.1 mg |
| c) | microcrystalline cellulose | 137.2 mg |
| | (e.g.: Avicel PH 102) | |
| d) | microcrystalline cellulose | 7.5 mg |
| | (e.g.: Avicel PH 101) | |
| e) | sodium carboxymethyl starch | 8.5 mg |
| f) | magnesium stearate | 1.7 mg |
| | | 170.0 mg |

a) is premixed with d) in a compulsory mixer. This mixture is admixed with b), c) and e) in the compulsory mixer. Subsequently f) is admixed in a free-fall mixer. The tabletting mixture is compressed to cores in a suitable tablet press.

II. Film layer

| | |
|---|---|
| g) Ethylcellulose | 3.71 mg |
| h) Dibutylsebacat | 1.25 mg |
| i) Lactose Monohydrat (mikronisiert) | 5.04 mg |
| | 180.0 mg |

g) and h) are solved in Ethanol. I) is suspendes in the clear solution.

The suspension is applied to the tablet cores obtained in I. in a suitable film-coating apparatus.

### Example 9

Film-Coated Tablets:

I. Production of the uncoated core:

| | | |
|---|---|---|
| a) | soraprazan | 10.0 mg |
| b) | sodium carbonate (anhydrous) | 5.1 mg |
| c) | microcrystalline cellulose | 137.2 mg |
| | (e.g.: Avicel PH 102) | |
| d) | microcrystalline cellulose | 7.5 mg |
| | (e.g.: Avicel PH 101) | |
| e) | sodium carboxymethyl starch | 8.5 mg |
| f) | magnesium stearate | 1.7 mg |
| | | 170.0 mg |

a) is premixed with d) in a compulsory mixer. This mixture is admixed with b), c) and e) in the compulsory mixer. Subsequently f) is admixed in a free-fall mixer. The tabletting mixture is compressed to cores in a suitable tablet press.

II. Film layer

| | |
|---|---|
| g) Celluloseacetat | 7.0 mg |
| h) Triethylcitrat | 2.34 mg |
| | 179.34 mg |

g) and h) are solved in a mixture of Aceton and purified water.

The suspension is applied to the tablet cores obtained in I. in a suitable film-coating apparatus. By using a suitable technology, e.g. a Laser apparatus, small wholes of 50 μm to 250 μm are produced into the coat.

### Example 10

Film-Coated Matrix Tablets:

| a) | soraprazan | 5.0 mg |
|---|---|---|
| b) | mannitol | 34.0 mg |
| c) | HPMC 2208 | 10.0 mg |
| d) | microcrystalline cellulose | 26.0 mg |
| | (e.g.: Avicel PH 101) | |
| e) | Uni Pure ® WG 220 | 3.0 mg |
| f) | basic buffer | 2.0 mg |
| Mass of granules | | 80.0 mg |
| g) | magnesium stearate | 0.25 mg |
| Mass of tablet core | | 84.25 mg |
| h) | film coating | 4.0 mg |

| | |
|---|---|
| Mass of film-coated tablet | 88.25 mg |

### Example 11

Film-Coated Matrix Tablets: 10 mg

| a) | soraprazan | 10.0 mg |
|---|---|---|
| b) | mannitol | 34.0 mg |
| c) | Eudragit RS | 10.0 mg |
| d) | microcrystalline cellulose | 21.0 mg |
| | (e.g.: Avicel PH 101) | |
| e) | Uni Pure ® WG 220 | 3.0 mg |
| f) | basic buffer | 2.0 mg |
| Mass of granules | | 80.0 mg |
| g) | magnesium stearate | 0.25 mg |
| Mass of tablet core | | 84.25 mg |
| h) | film coating | 4.0 mg |

| | |
|---|---|
| Mass of film-coated tablet | 88.25 mg |

### Example 12

Film-Coated Matrix Tablets:

| a) | soraprazan | 5.0 mg |
|---|---|---|
| b) | mannitol | 34.0 mg |
| c) | Ethylcellulose | 10.0 mg |
| d) | microcrystalline cellulose | 26.0 mg |
| | (e.g.: Avicel PH 101) | |
| e) | Uni Pure ® WG 220 | 3.0 mg |
| f) | sodium carbonate | 1.2 mg |
| Mass of granules | | 79.2 mg |
| g) | magnesium stearate | 0.25 mg |
| Mass of table core | | 83.45 mg |
| h) | film coating (PVA-based) | 3.55 mg |

| | |
|---|---|
| Mass of film-coated tablet | 87.00 mg |

### Example 13

A Hard Gelatine Capsule or a HPMC Capsule Contains:

One or Two Sustained Release Film-Coated Tablets with:

I. Production of the uncoated core:

| | | |
|---|---|---|
| a) | soraprazan | 2.5 mg |
| b) | sodium carbonate (anhydrous) | 1.275 mg |
| c) | microcrystalline cellulose | 34.3 mg |
| | (e.g.: Avicel PH 102) | |
| d) | microcrystalline cellulose | 1.875 mg |
| | (e.g.: Avicel PH 101) | |
| e) | sodium carboxymethyl starch | 2.125 mg |
| f) | magnesium stearate | 0.425 mg |
| | | 42.5 mg |

a) is premixed with d) in a compulsory mixer. This mixture is admixed with b), c) and e) in the compulsory mixer. Subsequently f) is admixed in a free-fall mixer. The tabletting mixture is compressed to cores in a suitable tablet press.

II. Film layer

| | |
|---|---|
| g) Surelease E 7-7050 | 3.5 mg |
| | 46.0 mg |

And One or Two Immediate Release Film-Coated Tablets with:

I. Production of the uncoated core:

| | | |
|---|---|---|
| a) | soraprazan | 2.5 mg |
| b) | sodium carbonate (anhydrous) | 1.275 mg |
| c) | microcrystalline cellulose | 34.3 mg |
| | (e.g.: Avicel PH 102) | |
| d) | microcrystalline cellulose | 1.875 mg |
| | (e.g.: Avicel PH 101) | |
| e) | sodium carboxymethyl starch | 2.125 mg |
| f) | magnesium stearate | 0.425 mg |
| | | 42.5 mg |

a) is premixed with d) in a compulsory mixer. This mixture is admixed with b), c) and e) in the compulsory mixer. Subsequently f) is admixed in a free-fall mixer. The tabletting mixture is compressed to cores in a suitable tablet press.

II. Film layer

| | |
|---|---|
| g) Opadry II green | 3.5 mg |
| | 46.0 mg |

Stability Testing

Triturations of soraprazan with different excipients including or excluding a basic excipient were manufactured, stored at 50° C. and analysed for impurities. The following results were obtained:

| | | | | Soraprazan, |
|---|---|---|---|---|
| | | | | Corn |
| | | | | Starch, |
| | | | | Mannit, |
| | | | | Magnesium |
| | | Soraprazan, | | Stearate, |
| | Soraprazan, | Magnesium | Soraprazan, | Disodium |
| Mixture | Mannit | Stearate | Corn Starch | Carbonate |
| Impurities | 5.29 | 5.01 | 6.67 | 3.76 |
| total | | | | |
| (AU %) | | | | |

| | Soraprazan, | |
|---|---|---|
| | Corn | Soraprazan, |
| | Starch, | Mannit |
| | Magnesium | Magnesium |
| | Stearate, | Stearate, |
| | Sodium | Sodium |
| | Hydrogen- | Hydrogen- |
| Mixture | carbonate | carbonate |
| Impurities | 3.68 | 3.74 |
| total (AU %) | | |

For triturations comprising a basic exipient a distinct lower impurity profile is observed.

### INDUSTRIAL APPLICABILITY

Proton pump antagonists and their salts have valuable pharmacological properties which make them industrially utilizable. They show in particular a pronounced inhibition of gastric acid secretion and an excellent gastrointestinal-protective effect in warm-blooded species, especially humans. The compounds according to the invention are distinguished in this connection by a high selectivity of effect, an advantageous duration of action, a particularly good enteral activity, the absence of substantial side effects and a high therapeutic index.

“Gastrointestinal protection” means in this connection the prevention and treatment of gastrointestinal disorders, especially gastrointestinal inflammatory disorders and lesions (such as, for example, gastric ulcer, duodenal ulcer, gastritis, hyperacidic or drug-related dyspepsia, heartburn and acid eructation, severe reflux oesophagitis, prophylaxis of recurrent reflux oesophagitis and of duodenal ulcer, reflux oesophagitis, Zollinger-Ellison syndrome, elimination of the pathogen Helicobacter pylori in combination with amoxicillin and clarithromycin or in combination with clarithromycin and metronidazole or with amoxicillin and metronidazole, long-term treatment for prophylaxis of recurrent severe forms of reflux oesophagitis. Prophylaxis and therapy of ulcers and gastroduodenal erosions induced by non-steroidal antiinflammatory drugs), which may be caused for example by microorganisms (e.g. Helicobacter pylori), bacteriotoxins, medicines (e.g. certain antiinflammatory and antirheumatic drugs), chemicals (e.g. ethanol), gastric acid or stress situations.

Owing to these properties, the dosage forms according to the invention containing a proton pump antagonist and/or a pharmacologically acceptable salt thereof are outstandingly suitable for use in human and veterinary medicine, being used in particular for the treatment and/or prophylaxis of disorders of the stomach and/or intestine.

The invention therefore further relates to the dosage forms according to the invention for use in the treatment and/or prophylaxis of the aforementioned disorders.

The invention also includes the use of the dosage forms according to the invention for the treatment and/or prophylaxis of the aforementioned disorders.

The dosage forms according to the invention can be combined with other medicaments, either in different combinations or in a fixed combination. Combinations worth mentioning in connection with the dosage forms according to the invention containing proton pump antagonists as active ingredients are those with antimicrobial active ingredients and those with NSAIDs (non steroidal anti inflammatory drugs). Particular mention should be made of the combination with antimicrobial agents like those employed to control the microbe Helicobacter pylori (H. pylori). Further examples which may be mentioned of combinations are: tranquilizers (for example from the group of benzodiazepines, e.g. diazepam), spasmolytics (e.g. bietamiverine or camylofin), anticholinergics (e.g. oxyphencyclimine or phencarbamide), local anesthetics (e.g. tetracaine or procaine), where appropriate also enzymes, vitamins or amino acids. Combinations of the compounds according to the invention with drugs which inhibit acid secretion should be particularly emphasized in this connection, such as, for example, antacids, H2 blockers (e.g. cimetidine, ranitidine), H+/K+-ATPase inhibitors (e.g. omeprazole, pantoprazole), or else with so-called peripheral anticholinergics (e.g. pirenzepine, telenzepine) and with gastrin antagonists with the aim of enhancing the main effect in an additive or superadditive sense and/or of eliminating or reducing the side effects.

Examples of suitable antimicrobial active ingredients (active against Helicobacter pylori) are described in EP-A-0 282 131. Examples which may be mentioned of antimicrobial agents suitable for controlling the microbe Helicobacter pylori are for example bismuth salts [e.g. bismuth subcitrate, bismuth subsalicylate, ammonium bismuth(III) potassium citrate dihydroxide, bismuth nitrate oxide, dibismuth tris(tetraoxodialuminate)], especially β-lactam antibiotics, for example penicillins (such as benzylpenicillin, phenoxymethylpenicillin, propicillin, azidocillin, dicloxacillin, flucloxacillin, oxacillin, amoxicillin, bacampicillin, ampicillin, mezlocillin, piperacillin or azlocillin), cephalosporins (such as cefadroxil, cefaclor, cefalexin, cefixime, cefuroxime, cefetamet, cefadroxil, ceftibuten, cefpodoxime, cefotetan, cefazolin, cefoperazone, ceftizoxime, cefotaxime, ceftazidime, cefamandole, cefepime, cefoxitin, cefodizime, cefsulodin, ceftriaxone, cefotiam or cefmenoxime) or other β-lactam antibiotics (e.g. aztreonam, loracarbef or meropenem); enzyme inhibitors, for example sulbactam; tetracyclines, for example tetracycline, oxytetracycline, minocycline or doxycycline; aminoglycosides, for example tobramycin, gentamicin, neomycin, streptomycin, amikacin, netilmicin, paromomycin or spectinomycin; amphenicols, for example chloramphenicol or thiamphenicol; lincomycins and macrolide antibiotics, for example clindamycin, lincomycin, erythromycin, clarithromycin, spiramycin, roxithromycin or azithromycin; polypeptide antibiotics, for example colistin, polymixin B, teicoplanin or vancomycin; gyrase inhibitors, for example norfloxacin, cinoxacin, ciprofloxacin, pipemidic acid, enoxacin, nalidixic acid, pefloxacin, fleroxacin or ofloxacin; nitroimidazoles, for example metronidazole; or other antibiotics, for example fosfomycin or fusidic acid. Administration of a reversible proton pump inhibitor together with the combination of a plurality of antimicrobial active ingredients is particularly worthy of mention in this connection, for example with the combination of a bismuth salt and/or tetracycline with metronidazole or the combination of amoxicillin or clarithromycin with metronidazole and amoxicillin with clarithromycin.

The dosage of the active ingredients in the dosage form according to the invention depends greatly on the nature of the proton pump antagonists used. A typical dosage for a proton pump antagonist as disclosed for example in WO-A-9418199 can be regarded as a daily dose of about 0.01 to about 20, preferably about 0.05 to 5, in particular 0.1 to 1.5, mg/kg of body weight, where appropriate in the form of a plurality of single doses. In the case of the compound soraprazan, examples of dosage forms according to the invention contain the proton pump antagonist in a dose of 2, 2.5, 5,10, 15,20 or 40 mg.

Antimicrobial active ingredients which may be emphasized are erythromycin, azithromycin, clarithromycin, clindamycin, rifampicin, ampicillin, mezlocillin, amoxicillin, tetracycline, minocycline, doxycycline, imipenem, meropenem, cefalexin, cefuroxime axetil, cefpodoxime proxetil, cefaclor, cefadroxil, ciprofloxacin, norfloxacin, ofloxacin and pefloxacin.

Antimicrobial active ingredients which may be particularly emphasized are clarithromycin and amoxicillin.

Combined administration for the purposes of the present invention mean fixed and, in particular, free combination, i.e. either the proton pump antagonist and the antimicrobial active ingredient are present here in one dosage unit, or the proton pump antagonist and antimicrobial active ingredient, which are present in separate dosage units, are administered in direct succession or at a relatively large interval in time, a relatively large interval in time meaning a time span not exceeding 24 hours. For use as separate dosage units, these are preferably provided in a common package. For example, the two dosage units are packaged together in blisters which are designed in respect of the relative disposition of the two dosage units, the labelling and/or colouring in a manner known per se so that the time that the individual components (dosage regimen) of the two dosage units should be taken are evident to the patient.

Dosage unit means, in particular, dosage forms such as tablets, coated tablets or pellets, and micro-tablets in capsules, the dosage form advantageously being designed so that the two active ingredient components (proton pump antagonist on the one hand and antimicrobial active ingredient on the other hand) are released or effectively made available to the body in such a way that an optimal active ingredient profile and thus profile of effect is achieved.

1. An oral dosage form for reversible proton pump inhibitors comprising an effective amount of a proton pump antagonist (APA) together with excipients, where the proton pump antagonist is stabilized in the dosage form by one or more basic excipients and which dosage form is an extended release dosage form.

2. The oral dosage form according to claim 1, wherein the basic excipient is present in finely divided form and thoroughly mixed with the proton pump antagonist.

3. The oral dosage form according to claim 1, wherein excipients which, on oral intake of the dosage form, bring about sustained release of the proton pump antagonist.

4. The oral dosage form according to claim 1, wherein the dosage form is selected from the group consisting of tablets, coated tablets, pellets, coated pellets, microtablets in capsules and granules in capsules.

5. The oral dosage form according to claim 4, which is a tablet or pellet containing a film coating providing sustained release of the proton pump antagonist.

6. The oral dosage form according to claim 4, which is a matrix tablet.

7. The oral dosage form according to claim 1, wherein part of the proton pump antagonist is in sustained release form and the other part is in immediate release form.

8. The oral dosage form according to claim 1, characterized in that it shows a release of active ingredient of of less than 90% in at least three hours in 0.1 N hydrochloric acid.

9. The oral dosage form according to claim 3, characterized in that one or more substances selected from the group consisting of fillers and polymers are present as excipients which bring a sustained release dosage form.

10. The oral dosage form according to claim 9, characterized in that at least one filler and at least one polymer are present.

11. The oral dosage form according to claim 10, characterized in that microcrystalline cellulose or/and a polyol is present.

12. The oral dosage form according to claim 1, characterized in that one or more further excipients selected from the group consisting of lubricants, aromas, colouring agents, flavourings and surface-active substances are present.

13. The oral dosage form according to claim 1, characterized in that the basic excipient is selected from the group consisting of sodium carbonate, calcium carbonate, magnesium carbonates, magnesium oxide, magnesium hydroxide, magnesium metasilicate aluminate, magnesium silicates, magnesium aluminate, hydrotalcite (synthetic), aluminium magnesium hydroxide, and calcium hydroxide, basic salts of amino acids, sodium hydroxide, trihydroxymethylaminomethane, trisodium citrate, disodium hydrogen phosphate, trisodium phosphate and mixtures thereof.

14. The oral dosage form according to claim 13, comprising sodium carbonate.

15. The oral dosage form according to claim 13, comprising disodium hydrogen phosphate, trisodium phosphate or buffer systems composed of disodium hydrogen phosphate and sodium hydroxide.

16. The oral dosage form according to claim 1, characterized in that a compound selected from the group consisting of AU-461, soraprazan (BYK61359), DBM-819, KR-60436, T-330, YH-1885, YJA-20379-8 and 2,3-dimethyl-8-(2-ethyl-6-methylbenzylamino)imidazo[1,2-a]pyridine-6-carboxamide is present as reversible proton pump inhibitor.

17. The oral dosage form according to claim 16, characterized in that (7R,8R,9R)-2,3-dimethyl-8-hydroxy-7-(2-methoxyethoxy)-9-phenyl-7,8,9,10-tetrahydroimidazo[1,2-h][1,7]naphthyridine (INN soraprazan) or a pharmacologically acceptable salt and/or hydrate thereof is present as proton pump antagonist.

18. The oral dosage form according to claim 1, comprising (7R,8R,9R)-2,3-dimethyl-8-hydroxy-7-(2-methoxyethoxy)-9-phenyl-7,8,9,10-tetrahydroimidazo[1,2-h][1,7]naphthyridine (INN soraprazan) or a pharmacologically acceptable salt and/or hydrate thereof as proton pump antagonist, sodium carbonate as basic excipient and microcrystalline cellulose, sodium carboxymethyl starch and magnesium stearate as excipients.

19. The oral dosage form according to claim 18, which is a film coated tablet.

20. The oral dosage form according to claim 19, which comprises a coloured film coating.
